(19) United States
(12) Patent Application Publication
(10) Pub. No.: US 2021/0272677 A1
(43) Pub. Date: Sep. 2, 2021

(54) SYSTEM AND METHOD FOR PATIENT VERIFICATION

(71) Applicant: NEW YORK UNIVERSITY, New York, NY (US)

(72) Inventor: David Barbee, Long Island City, NY (US)

(21) Appl. No.: 17/186,568

(22) Filed: Feb. 26, 2021

Related U.S. Application Data
(60) Provisional application No. 62/982,812, filed on Feb. 28, 2020.

(51) Int. Cl. G16H 20/40; H04L 29/08; G16H 40/67; H04L 29/06

(52) U.S. Cl. CPC G16H 20/40; H04L 63/08; G16H 40/67; H04L 67/36

(57) ABSTRACT

A system for subject verification comprises at least one computing device, at least one input device communicatively connected to the at least one computing device, at least one electromechanical control interface communicatively connected to the at least one computing device, configured to deliver at least one electrical signal to a treatment delivery system, and a user interface executing on the at least one computing device, providing a set of prompts to a user for verifying an identity of a subject based in part on inputs received from the at least one input device, and configured to send a communicative signal to the electromechanical control interface when the subject's identity is verified. Methods of verifying subject identity are also described.

### CROSS-REFERENCE TO RELATED APPLICATIONS

This application claims priority to U.S. Provisional Patent Application No. 62/982,812, filed on Feb. 28, 2020, incorporated herein by reference in its entirety.

### BACKGROUND OF THE INVENTION

Patient verification is a critical component to safe delivery of radiation to the appropriate patient. A patient timeout (PTO) is defined as a brief pause by the entire clinical team immediately before starting a medical procedure to confirm the correct patient, procedure, and site. Typical timeouts may involve all the immediate members of the procedure team: the individual performing the procedure, anesthesia providers, circulating nurse, operating room technician, and other active participants who will be participating in the procedure from the beginning. The performance of the timeout is documented where possible, with timestamps and other validation methods to confirm it was performed.

The American College of Radiology (ACR) recommends performing a patient time out (PTO) prior to initiating radiation treatment on a treatment delivery system (TDS), verifying a minimum of two forms of identification, e.g. patient name, patient date of birth, patient medical record number, patient photo, patient treatment site. The ACR further recommends that PTO should be conducted by two personnel and should be recorded as non-editable timestamps in the patient's chart.

Newer TDSs and oncology information systems often lack timeout integration or implement them in ways that are not clinically useful or meaningful, often lacking signatures and timestamps. Some existing systems provide alerts or software-based interlocks, or variously force the provider to verify all other treatment accessories, or fail to generate timestamps. No existing system provides a hard stop interlock on the machine, making radiation delivery impossible until the identity of the patient is verified. Additionally, due to differences in technology across vendors, there is no unified, standardized method for performing signed and dated timeouts that control hard stop interlocks on treatment machines.

Thus, there is a need in the art for an improved patient timeout system for use with clinical therapeutic or diagnostic machines, which allows for a mechanical or electrical stop to be engaged until the timeout process is complete. The present invention satisfies this need.

### SUMMARY OF THE INVENTION

In one aspect, a system for subject verification comprises at least one computing device, at least one input device communicatively connected to the at least one computing device, at least one electromechanical control interface communicatively connected to the at least one computing device, configured to deliver at least one electrical signal to a treatment delivery system, and a user interface executing on the at least one computing device, providing a set of prompts to a user for verifying an identity of a subject based in part on inputs received from the at least one input device, and configured to send a communicative signal to the electromechanical control interface when the subject's identity is verified.

In one embodiment, the treatment delivery system is selected from the group consisting of a medical linear accelerator and a radioactive isotope therapy machine. In one embodiment, the at least one computing device comprises a first computing device executing the user interface and a second computing device communicatively connected to the electromechanical control interface. In one embodiment, the at least one electrical signal is delivered to at least one relay. In one embodiment, the at least one computing device comprises a single-board computer.

In one embodiment, the at least one computing device is communicatively connected to at least one external computing device comprising a database. In one embodiment, the at least one external computing device comprises an oncology information system. In one embodiment, the at least one external computing device comprises a database server configured to store timeout event data on a non-transitory computer-readable medium. In one embodiment, the at least one external computing device comprises an authentication server. In one embodiment, the authentication server is an active directory server.

In another aspect, a method of verifying subject identity for treatment with a treatment device comprises acquiring and displaying information about a subject to a user interface, indicating to an external system that a procedure is in progress, accepting a verification of the identity of the subject based on the displayed information, accepting at least one sign-off on the verification, and sending a signal to a treatment delivery system when the verification is complete, to enable a function of the treatment delivery system.

In one embodiment, the method further comprises transmitting timestamped data about the verification to an external database. In one embodiment, the method further comprises accepting a biometric form of verification selected from the group consisting of facial recognition, palm scanning, retinal scanning, and fingerprint scanning. In one embodiment, the method further comprises displaying at least one alert about the procedure on the user interface. In one embodiment, the method further comprises displaying at least one status message about the procedure on the user interface.

In another aspect, a method of verifying subject identity for treatment with a treatment device comprises querying a clinical database for information about a subject via a user interface, comparing displayed subject information to a subject, verifying the identity of the subject, providing at least one electronic signature via the user interface, and sending a signal to a treatment delivery system via the user interface, to enable a function of the treatment delivery system.

In one embodiment, the information about the subject is selected from the group consisting of an image of the subject, a name of the subject, a date of birth of the subject, a barcode, a palm print, a fingerprint, and a retinal scan. In one embodiment, the at least one electronic signature is at least two electronic signatures. In one embodiment, the signal is an electrical signal configured to actuate a relay. In one embodiment, the method further comprises authenticating against an external authentication database.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of an exemplary treatment delivery system;

FIG. 2 is an exemplary system diagram of a PTO system;

FIG. 3A is an exemplary system diagram of a PTO system;

FIG. 3B is a schematic of an electromechanical control interface;

FIG. 4 is an exemplary user interface for use with a PTO system; and

FIG. 5 is a method of the disclosure.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

As used herein, the term “moding up” means to send patient treatment information to a treatment delivery system.

As used herein, the term “treatment delivery system” (abbreviated as TDS) includes any or a combination of multiple treatment modalities, including but not limited to a photon/electron/heavy ion accelerator, radioactive source machine (GammaKnife or HDR Remote Afterloaders), or any system configured to deliver a treatment or diagnostic to a subject, the treatment or diagnostic in some embodiments comprising one or more forms of radiation.

The terms “patient” and “subject” are used interchangeably herein, and refer to any individual being subjected to a treatment or evaluation in the context of the present disclosure.

In various aspects, a system of the present invention comprises one or more computing devices executing software to verify patient identity, record the identity and the patient information, and upon verification, send an electrical signal to an electromechanical actuator to unlock or otherwise enable a subsystem of a treatment delivery system.

Systems of the invention may include one or more input devices for accepting patient information. Suitable input devices include, but are not limited to, keyboards, mice, cameras, fingerprint scanners, palm scanners, barcode scanners, facial recognition systems, retinal scanners, iris scanners, microphones optionally including voice recognition systems, or the like. As contemplated herein, a system of the invention may query one or more input devices to obtain information about a patient physically present near a treatment device using a system of the invention.

In some embodiments, some or all patient data may be entered manually by a clinician using a keyboard/mouse interface. In some embodiments, some or all patient data may be acquired by scanning a barcode or other identifying marker, for example a barcode on a patient bracelet or other garment. In some embodiments, acquired patient data may include one or more images of the patient's face, taken by one or more cameras simultaneously. In some embodiments, acquired patient data may include one or more fingerprints, hand prints, foot prints, retinal scans, voice recordings, video recordings of gait, or any other biometric identification able to be acquired by a computing device.

A system may additionally include one or more interfaces to external systems, including electronic medical records (EMR) or patient information databases, clinician databases, oncology information systems (OIS), or the like. Such interfaces may be used for example to acquire stored information about a patient, for example stored biometric or identifying data, to compare with biometric and/or identifying data acquired at the treatment device.

A system may include a logging database interface for recording acquired patient information and clinician sign-off information, along with timestamps and any other ancillary data desired for verification purposes. Such a logging database interface may provide formal documentation that a PTO was performed as required before the treatment.

Finally, a system may include an electromechanical control interface, for example a general-purpose IO (GPIO) or other electrical signaling interface to either actuate a physical actuator or control a relay, for example a solid-state relay (SSR) to enable hard control of an interlock on a treatment delivery system. In one embodiment, an electromechanical control interface may comprise an electrical signal opening a relay that, when open, prevents a radiation beam or other potentially hazardous clinical system in the TDS from being activated. In other embodiments, the relay when closed may lock a door, preventing individuals from accessing the room or chamber where a radiation beam or treatment system is applied to the patient. In some embodiments, an electromechanical control interface may be electrically connected to an existing switch on the TDS, for example a last man out switch, a door switch, a relay junction box, or other lock out system.

In some embodiments, a system may include one or more additional sensors to be used in addition to or in place of a last man out switch. Exemplary sensor configurations include, but are not limited to, radiation sensors to indicate beam on or measure energy, and one or more motion, proximity, pressure, or threshold sensors to verify that the number of personnel entering matches the number of personnel leaving (thus verifying that all personnel have left the TDS before treatment commences).

Different electromechanical control interfaces may be configured to stop/lock out all beam types, imaging-only beams, or treatment-only beams, allowing flexibility to ensure patients receive the prescribed procedure (e.g. pre-treatment imaging films only without treatment on day 1 of care).

In some embodiments, an electromechanical control interface may be configured to fail open, in other words to be configured to disable the TDS or radiation beam or treatment system in the event of an error or power down.

Advantageously, a system of the invention may in some embodiments be independent from the one or more TDSs with which the system interfaces. In other words, the system may have no software or data interfaces with any software systems running on a computing device integrated into the TDS. By avoiding using a software interface, a system of the invention may be easily compatible with TDSs made by a variety of different manufacturers without significant changes to configuration.

A system of the invention may in some embodiments be used even in a pre-treatment verification appointment, for example in a preliminary appointment where a subject is introduced into the TDS but where no treatment is intended. Pre-treatment verification appointments may in some embodiments be an option in a user interface, wherein the user interface does not release the interlock via the electromechanical control interface when an appointment is a films only appointment.

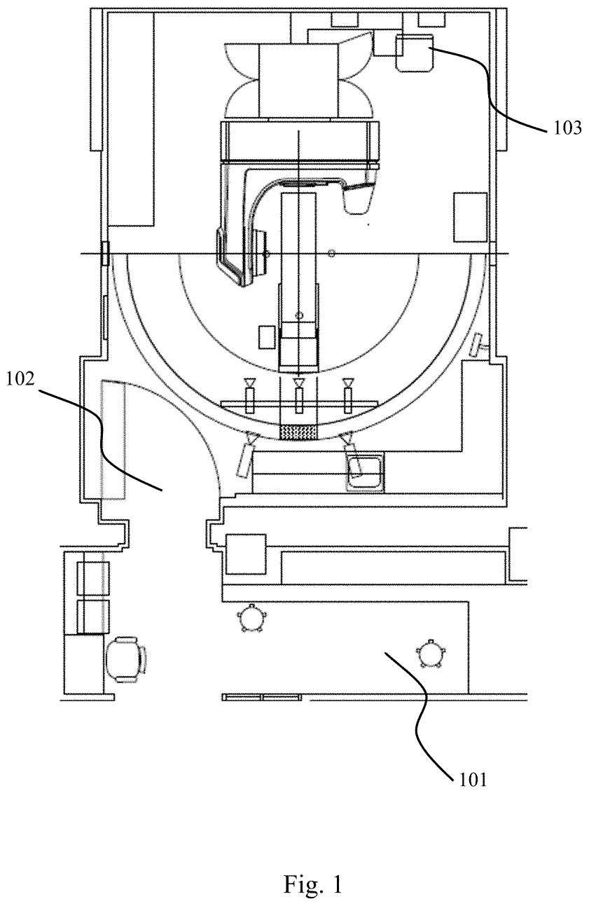

An exemplary TDS is shown in FIG. 1. The TDS includes machine control area 101, timeout area 102, and relay junction box 103. Various exemplary embodiments of a system of the invention are described with reference to the layout in FIG. 1.

In one embodiment, a first computing device is positioned at the timeout area 102, including one or more wires (not shown) extending from the first computing device to the relay junction box 103 forming the electromechanical control interface. The wires may form a connection for example to a relay input from a GPIO on the first computing device. A second computing device may be positioned at machine control 101 for acquiring additional signoffs—both the first computing device and the second computing device may be controlled from a single user interface positioned at the timeout area via a KVM (keyboard, video, mouse) connection or some other method, for example a single touchscreen interface.

In one embodiment, a first computing device is positioned at the machine control area 101, with the user interface positioned at the patient timeout area 102. The user interface may be connected to the first computing device via a KVM or other networked/long run user interface connection. Similarly to the first embodiment, one or more wires extend from the first computing device to the relay junction box 103 for the electromechanical control interface.

In one embodiment, a first computing device is positioned at the relay junction box 103, with a shorter electrical connection from the first computing device to the relay junction box for the electromechanical control interface. The first computing device may accept software commands remotely from one or more remote computing devices. In this embodiment, a second computing device may be positioned in the machine control area 101, with a user interface positioned in timeout area 102. A user in the timeout area 102 may issue commands via the user interface to the second computing device, which then relays or translates those commands to the first computing device positioned near the relay junction box 103. The first computing device may then send an electrical signal to the RJB 103, for example opening or closing one or more relays 103.

In one embodiment, a first computing device may be positioned at the timeout area 102, with the first computing device communicatively connected to one or more input devices, for example biometric scanners, cameras, or any other input devices as contemplated herein. The first computing device may have additional input interfaces via KVM positioned at machine control 101. A second computing device may be positioned at the RJB 103, communicatively connected to the first computing device and configured to control one or more relays for performing the electromechanical control interface functions.

Although the above examples are described as distinct systems, it is understood that the present disclosure contemplates combinations of the various embodiments and configurations described herein, and that certain systems may include one or more features of one embodiment and one or more features of other embodiments. In some embodiments, one or more of the computing devices is an embedded computing device or small form factor computer, for example a Raspberry Pi or other single-board computer.

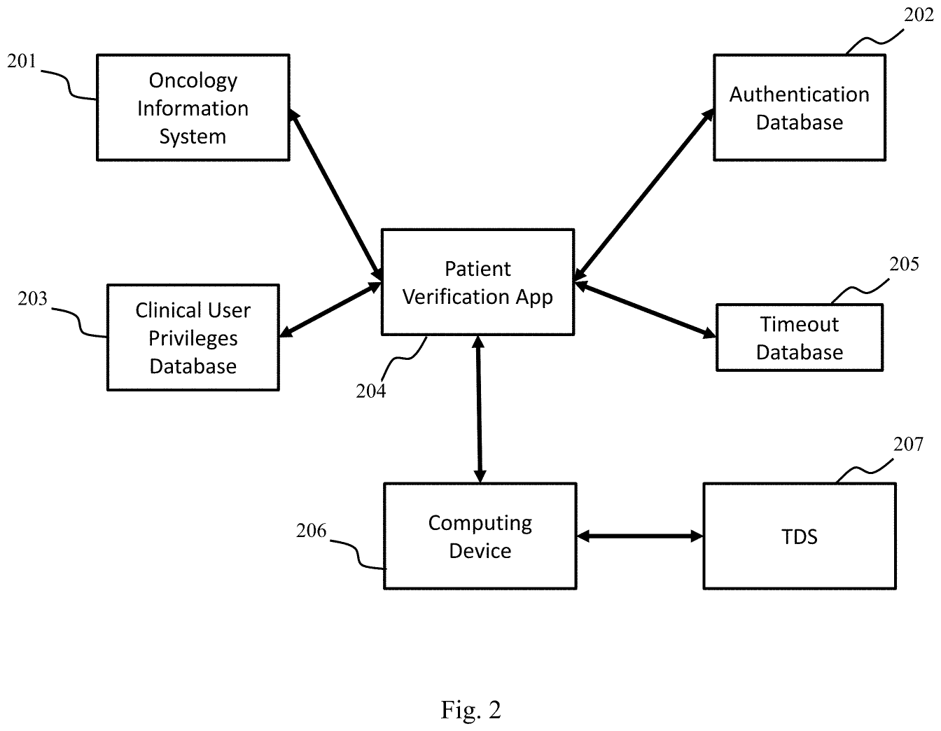

An overall system diagram of an exemplary embodiment is shown in FIG. 2. The Oncology Information System (OIS) 201 may be used to pull moded up patient and information to the patient verification application 204. The verification application 204 provides the primary user interface for the timeout process. The authentication database 202 may be used to authenticate the user of the patient verification app 204, who may in some embodiments be a clinician. In some embodiments, the authentication database 202 may comprise an active directory server or any other equivalent user authentication database.

The clinical user privileges database 203 may in some embodiments include user specific clinical privileges, including for example privileges to execute the clinical procedure at the TDS, privileges necessary to change one or more settings on the TDS, or privileges necessary to sign off on the clinical procedure at the TDS. The clinical privileges may in some embodiments be specific to the TDS, specific to the patient, or both. In some embodiments, the clinical user privileges database 203 may be incorporated into the authentication database 202, or vice versa. The timeout database 205 may be used to store the timestamped timeout logs, as discussed herein. The interface between the patient verification app 204 and the timeout database 205 may be any suitable database interface.

Finally, the patient verification app 204 is communicatively connected to the computing device 206, which in turn is electrically connected to the TDS 207 via for example a GPIO which opens and closes a relay in an RJB attached to the TDS for a hard interlock. In some embodiments, the computing device 206 may additionally receive one or more electrical signals from the TDS or RJB, for example open/closed indicator signals, voltage levels related to individual device power status, or serial data. Such signals may be relayed back to the patient verification app 204 for presentation to the user, and/or stored in the timeout database 205. In some embodiments, some or all of the functions of the patient verification app 204 are executed on computing device 206, while in other embodiments the patient verification app 204 executes on a separate computing device communicatively connected to the computing device 206, for example via a wired or wireless network interface, Bluetooth interface, or the like.

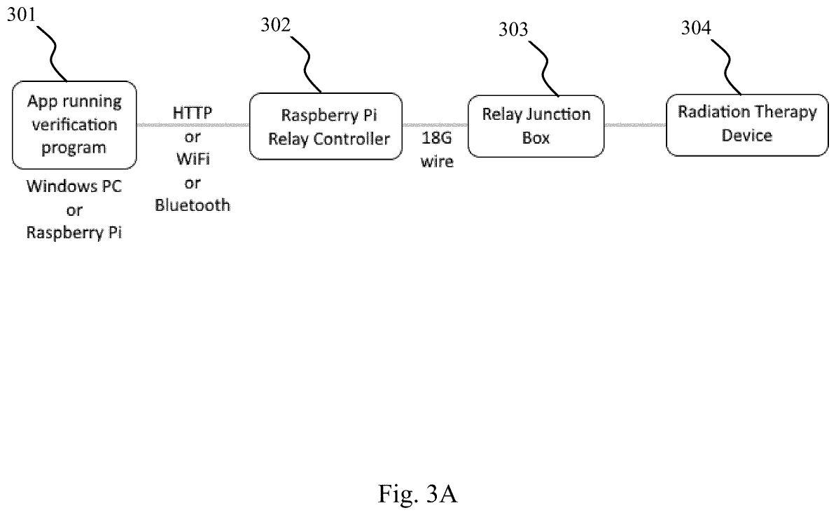

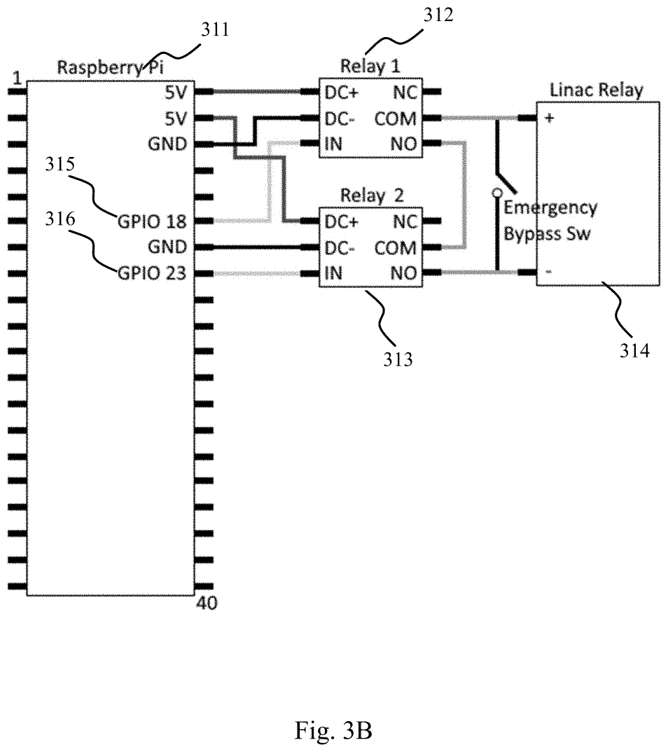

Another exemplary overall system diagram is shown in FIG. 3A. The depicted diagram shows first computing device 301 which may in some embodiments be a single board computer or a workstation/laptop. The first computing device 301 may run software performing verification methods as described herein. The first computing device 301 may be connected to a second computing device 302, which may also be a single board computer or another suitable computing device. The connection between first computing device 301 and second computing device 302 may be any wired or wireless data connection, including but not limited to HTTP, WiFi, or Bluetooth. The second computing device 302 is then connected to one or more relays in a relay junction box 303, using one or more wires. The depicted embodiment recites 18 gauge wire, but any suitable gauge may be used, for example as recommended by a TDS vendor. The relay junction box 303 is in turn electrically connected to the therapy device 304, and individual relays in relay junction box 303 may be controlled to enable or disable functions of the therapy device 304.

A schematic of a detail view of the system (specifically elements 302 and 303) is shown in FIG. 3B. In the depicted embodiment, a Raspberry Pi 311 is used as the second computing device 302. Two GPIOs 315 and 316 are electrically connected to the control inputs of two intermediate relays 312 and 313. The normally-open (NO) output of the intermediate relay 312 is connected to the common output (COM) of intermediate relay 313, while the COM of relay 312 is connected to the positive input of relay 314, which is positioned in relay junction box 303 and controls functionality of a treatment device. The NO output of intermediate relay 313 is connected to the negative input of relay 314.

When both GPIOs 315 and 316 are in the active state (which may be high or low depending on whether relays 312 and 313 are active-high or active-low relays), both relays close, and the connection between the COM and NC outputs of relays 312 and 313 are both shorted. The effect is to short the two inputs to relay 314, activating relay 314 and clearing the interlock.

In some embodiments, a QA bypass mode may be available for non-treatment settings for use in routine quality assurance testing of the TDS or pre-treatment plan delivery verification. In these situations, test plans are moded up on the TDS, to be delivered to quality assurance devices, wherein PTO is not possible. QA bypass circumvents PTO and sends messages to close the relay and release the interlock. A PTO system may include a check for user privileges prior to allowing the interface to manually actuate the hard interlocks via a QA bypass mode.

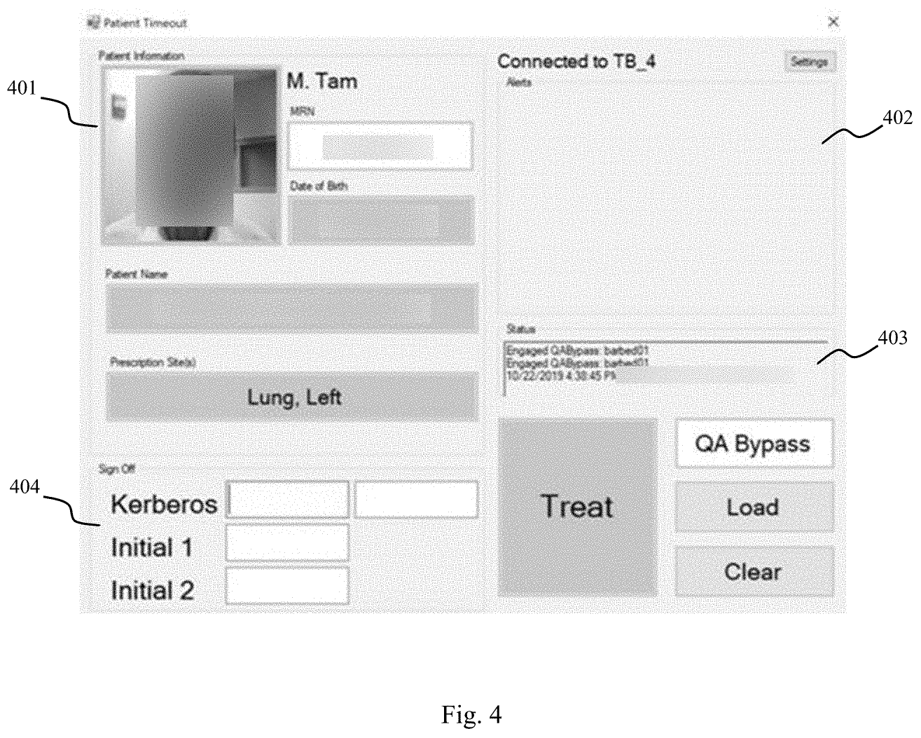

An exemplary user interface for the patient verification app is shown in FIG. 4, and is explained in greater detail below with regard to various methods of verifying patients for treatment with a treatment device as contemplated herein.

In some embodiments, the present disclosure also includes methods of verifying patients for treatment with a treatment device. A user (who may in some embodiments be a clinician) may first mode up a patient, presenting the user with biographical data about the subject in a first section 401. Biographical data may include the patient's name, one or more images of the patient, the patient's date of birth, a unique ID number, barcode, or the like. The user may also be presented with clinical information, including details about the procedure to be performed or details about the treatment site as acquired from an OIS. In some embodiments, as an effect of the user moding up the patient, that patient's procedure may be placed into an “in progress” status on the OIS schedule.

In some embodiments, the patient information may be presented with one or more alerts in an alerts area of a user interface 402. Alerts as understood herein may include status messages or other data included for the patient and/or procedure scheduled in the OIS, related to the moded-up patient. Example alerts include, but are not limited to on-treatment visit with physician, films only without treatment, unapproved prior images, weekly physics check needed, alerts entered into the OIS, plan modification, weight loss, multiple isocenters, collision risk, image-guidance match instructions, setup instructions, etc.

After the patient information and data have been loaded and presented on the user interface, a patient may be brought into the timeout area (for example 102 in FIG. 1) for the timeout, requiring a minimum number of forms of identification, for example at least two forms, at least three forms, at least four forms, or any other suitable number of forms of identification as desired or required by clinical procedure. In some embodiments, verification may be performed manually, for example by a clinician/user asking the patient for name, date of birth, etc. In some embodiments, a physician may click one or more fields in the user interface as each manual verification is performed, and a visual indication (for example a green background) may be used to indicate that the biographical information has been confirmed.

In other embodiments, some elements of verification (facial recognition, barcode scanning, palm scanning, fingerprint scanning, etc.) may be performed automatically, for example by pressing the patient's hand against a sensor or aiming one or more cameras at the patient's face.

In some embodiments, the timeout process may further comprise further checks of conditions in the OIS. In such embodiments, if one or more conditions are not met as detected in the OIS, the PTO system may prevent the patient from being treated. Examples of suitable conditions to be checked include, but are not limited to, unapproved documents or tasks (for example simulation orders, consult notes), unapproved films/imaging, weekly physics chart check not completed, for example within a billing cycle, physician on treatment visit (OTV) not performed, for example within a billing cycle, physics patient specific QA missing or not approved, pregnancy or other dependent diagnostic test status not documented in chart, unreviewed dosimetry measurements (diodes) or lab results, or unreviewed communications from patient or other physician.

In various embodiments, one or more of the aforementioned conditions may prevent the patient from being treated, and/or may require additional action from a clinician, administrator, or other professional in order to bypass. In some embodiments, the system may generate one or more notifications related to an unmet condition requiring sign-off by one or more professionals. In some embodiments, different conditions may be assigned different priority or security levels, requiring different levels of authorization to override or bypass. In some embodiments, the system may generate further instructions or post-treatment requirements for the patient (e.g. patient to see physician after treatment).

In some examples, conditions as outlined above may be configured for patient safety. In some embodiments, some conditions may alternatively or additionally be configured to prevent lost charge capture, i.e. billing compliance.

In some embodiments, the timeout process may further act as a final constancy check, for example comparing the loaded plan energy or dose/fraction with prescription values from the OIS, an EMR, or other data source.

Once the timeout process has been completed, one or more clinician users may be required to sign off on the completed PTO. The PTO system may verify user sign off credentials using for example a clinical user privileges database or an authentication database. Optionally, the PTO system may check the clinician's user rights in the OIS to ensure they have rights to treat patients. Clinician sign-offs may be accomplished and/or displayed via a sign-off region 404 of an exemplary user interface. In some embodiments, all clinicians must sign off via the same user interface, while in other embodiments, via for example a KVM link, one or more required clinicians may sign off remotely.

After the sign-off process is completed, in some embodiments, some or all PTO verified parameters may be written to a timeout database including therapist IDs, associated time stamps, associated OIS schedule identifiers. Additional data and parameters that may be written to the timeout database include, but are not limited to immobilization devices, additional personnel present, biometric similarity measures, treatment machine, mode up time, close out time. The data stored in the timeout database may be reviewable, for example via a user interface, and may be exportable as a report for entering into an OIS for documentation of all PTO for each patient treatment.

The PTO system may then send an electrical or data signal to a GPIO or other interface to enable a function of a TDS. In some embodiments, the electrical or data signal opens or closes one or more relays in a RJB and/or clears associated interlocks determined by PTO logic allowing for one of a set of operation modes for the TDS, including but not limited to, delivery of all beams, imaging only beams, treatment only beams, or delivery from radioactive source.

The PTO system may then begin a recurring query to the OIS to ensure the currently moded-up patient matches the current PTO authorized patient. Status messages may be displayed for the user for example in a status area 403. Exemplary status messages include, but are not limited to, device connection status, authorization status, cleared patients, or moded up patients.

In some embodiments, a PTO method may include one or more of various possible steps which result in the interlock being reasserted (e.g. one or more beams being turned off). Exemplary interlock steps include a patient completing treatment (at which point a “complete” status may be sent to an OIS from the TDS and the PTO detects that no patient is moded up via the recurring query discussed above); a user may hit a “clear” or “beam off” button to abort or end a treatment procedure; a patient mode up on the TDS may be cleared, which will remove the “in progress” status from the OIS; or a different patient may be moded up on the TDS (“in progress” on the OIS) and the new patient may then be loaded on the PTO interface.

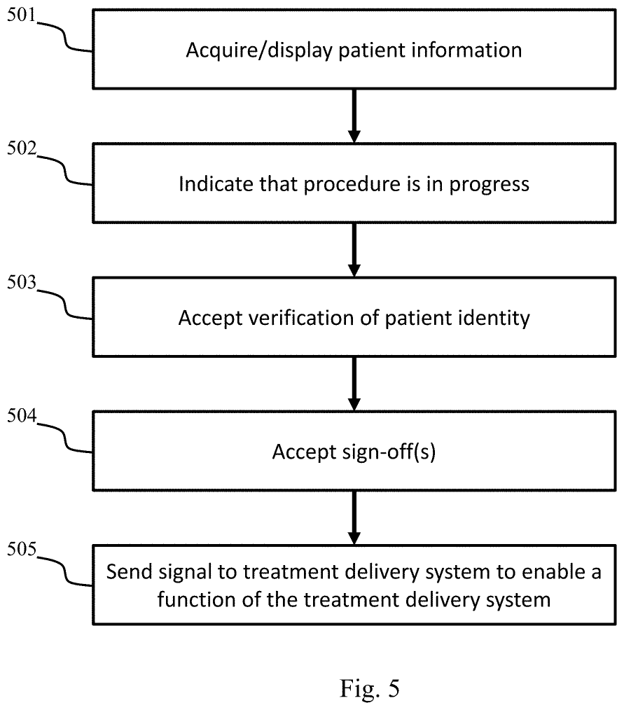

With reference to FIG. 5, a method of verifying patients for treatment with a treatment device is shown. The depicted method comprises the steps of acquiring and displaying information about a patient to a user interface in step 501, indicating to an external system that a procedure is in progress in step 502, accepting a verification of the identity of the patient based on the displayed information in step 503, accepting at least one sign-off on the verification in step 504, and sending a signal to a treatment delivery system when the verification is complete, to enable a function of the treatment delivery system in step 505.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system for subject verification, comprising:
at least one computing device;
at least one input device communicatively connected to the at least one computing device;
at least one electromechanical control interface communicatively connected to the at least one computing device, configured to deliver at least one electrical signal to a treatment delivery system; and
a user interface executing on the at least one computing device, providing a set of prompts to a user for verifying an identity of a subject based in part on inputs received from the at least one input device, and configured to send a communicative signal to the electromechanical control interface when the subject's identity is verified.

2. The system of claim 1, wherein the treatment delivery system is selected from the group consisting of a medical linear accelerator and a radioactive isotope therapy machine.

3. The system of claim 1, wherein the at least one computing device comprises a first computing device executing the user interface and a second computing device communicatively connected to the electromechanical control interface.

4. The system of claim 1, wherein the at least one electrical signal is delivered to at least one relay.

5. The system of claim 1, wherein the at least one computing device comprises a single-board computer.

6. The system of claim 1, wherein the at least one computing device is communicatively connected to at least one external computing device comprising a database.

7. The system of claim 6, wherein the at least one external computing device comprises an oncology information system.

8. The system of claim 6, wherein the at least one external computing device comprises a database server configured to store timeout event data on a non-transitory computer-readable medium.

9. The system of claim 6, wherein the at least one external computing device comprises an authentication server.

10. The system of claim 9, wherein the authentication server is an active directory server.

11. A method of verifying subject identity for treatment with a treatment device, comprising:
acquiring and displaying information about a subject to a user interface;
indicating to an external system that a procedure is in progress;
accepting a verification of the identity of the subject based on the displayed information;
accepting at least one sign-off on the verification; and
sending a signal to a treatment delivery system when the verification is complete, to enable a function of the treatment delivery system.

12. The method of claim 11, further comprising transmitting timestamped data about the verification to an external database.

13. The method of claim 11, further comprising accepting a biometric form of verification selected from the group consisting of facial recognition, palm scanning, retinal scanning, and fingerprint scanning.

14. The method of claim 11, further comprising displaying at least one alert about the procedure on the user interface.

15. The method of claim 11, further comprising displaying at least one status message about the procedure on the user interface.

16. A method of verifying subject identity for treatment with a treatment device, comprising:
querying a clinical database for information about a subject via a user interface;
comparing displayed subject information to a subject;
verifying the identity of the subject;
providing at least one electronic signature via the user interface; and
sending a signal to a treatment delivery system via the user interface, to enable a function of the treatment delivery system.

17. The method of claim 16, wherein the information about the subject is selected from the group consisting of an image of the subject, a name of the subject, a date of birth of the subject, a barcode, a palm print, a fingerprint, and a retinal scan.

18. The method of claim 16, wherein the at least one electronic signature is at least two electronic signatures.

19. The method of claim 16, wherein the signal is an electrical signal configured to actuate a relay.

20. The method of claim 16, further comprising authenticating against an external authentication database.
